(19) United States
(12) Patent Application Publication
(10) Pub. No.: US 2019/0219512 A1
(43) Pub. Date: Jul. 18, 2019

(54) DEVICE, PROCEDURE AND SYSTEM FOR DETECTING BACTERIAL PATHOGENS INCLUDING METHICILLIN-RESISTANT STAPHYLOCOCCUS AUREUS OR CLOSTRIDIUM DIFFICILE

(71) Applicants: Clifford H. Kern, III, Metairie, LA (US); Edward M. Yokley, Anderson, SC (US); William Tison Wyatt, Marshall, NC (US); Morton Greene, Las Vegas, NV (US)

(72) Inventors: Clifford H. Kern, III, Metairie, LA (US); Edward M. Yokley, Anderson, SC (US); William Tison Wyatt, Marshall, NC (US); Morton Greene, Las Vegas, NV (US)

(21) Appl. No.: 16/246,172

(22) Filed: Jan. 11, 2019

Related U.S. Application Data
(60) Provisional application No. 62/709,278, filed on Jan. 12, 2018.

(51) Int. Cl. G01N 21/77; G01N 33/02; A61K 39/085

(52) U.S. Cl. CPC G01N 21/77; C07K 16/1271; A61K 39/085; G01N 33/02

(57) ABSTRACT

A bio-sensor device for the electro-chemical detection of a bacterial pathogen, the device including a sample chamber and an electronic data module. The sample chamber includes electrical probes to detect pathogenic antigens in a sample containing the bacterial pathogen. The electrical probes detect a reaction voltage corresponding to an antigen-antibody reaction occurring when the pathogenic antigens come into contact with an antibody specific for pathogenic antigens present in a reaction medium in the sample chamber and contacted by the electrical probes. The electronic data module detects and processes electrical signals from the conductive electrical probes corresponding to an amount of the antigen present in the sample, wherein the reaction voltage is detected at the time of the reaction.

### CROSS-REFERENCE TO RELATED APPLICATION

This application claims the benefit of U.S. Provisional Application Ser. No. 62/709,278, filed by Clifford H. Kern III, et al. on Jan. 12, 2018, entitled, “TESTING DEVICE, PROCEDURE AND SYSTEM FOR METHICILLIN-RESISTANT STAPHYLOCOCCUS AUREUS AND CLOSTRIDIUM DIFFICILE,” commonly assigned with this application and incorporated herein by reference.

### TECHNICAL FIELD

This application is directed, in general, to a biosensor device and, more specifically, to a bio-sensor device for the electro-chemical detection of a bacterial pathogen.

### BACKGROUND

The rapid detection of bacterial pathogens, such as Staphylococcus aureus and Clostridium difficle, is important to the early diagnosis and treatment of patients, mitigating the spread of such pathogens and confirming that surfaces potentially contaminated with such pathogens have been de-contaminated.

### SUMMARY

One aspect provides a bio-sensor device for the electro-chemical detection of a bacterial pathogen. The device includes a sample chamber and an electronic data module. The sample chamber includes electrical probes to detect pathogenic antigens in a sample containing the bacterial pathogen. The electrical probes detect a reaction voltage corresponding to an antigen-antibody reaction occurring when the pathogenic antigens come into contact with an antibody specific for pathogenic antigens present in a reaction medium in the sample chamber and contacted by the electrical probes. The electronic data module detects and processes electrical signals from the conductive electrical probes corresponding to an amount of the antigen present in the sample, wherein the reaction voltage is detected at the time of the reaction.

In some such embodiments, the bacterial pathogen is one of methicillin-Resistant Staphylococcus aureus, Clostridium difficle or a combination of methicillin-Resistant Staphylococcus aureus and Clostridium difficle. In some such embodiments, the pathogen is detected from direct testing of a, swab, or washings from a surface. In any such embodiments, the surface can be an epidermis of an organism, a potentially contaminated non-biologic surface including a counter-top or synthetic athletic playing surface, wound dressing.

In any such embodiments, the device can be configured as a real-time detection device for detecting the presence of the pathogenic antigens, and the real-time detection device can be self-contained and field-applicable, not requiring external equipment or highly trained laboratory personnel. In some such embodiments, the electrical probes of the real-time detection device can be configured to respond to electrochemical antigen-antibody events corresponding to the antigen-antibody reaction within 60 seconds of the sample containing the pathogenic antigens and the antibody-containing reaction medium in the sample chamber becoming in contact with each other. In some such embodiments, the real-time detection device can be configured for direct electrochemical reaction detection of the antigen-antibody reaction. In some such embodiments, such real-time detection device is not sensitive to detection of reaction products of the antigen-antibody reaction.

In any such embodiments, the antibody specific for the pathogenic antigens can be coated onto one or more of the sensing electrodes. In any such embodiments, the antibody specific for the pathogenic antigens can be coated or adsorbed onto a passive filler located within the sample chamber. In any such embodiments, the sample chamber includes a port for introducing a liquid reagent containing the antibody specific for the pathogenic antigens.

### BRIEF DESCRIPTION

Reference is now made to the following descriptions taken in conjunction with the accompanying drawings, in which:

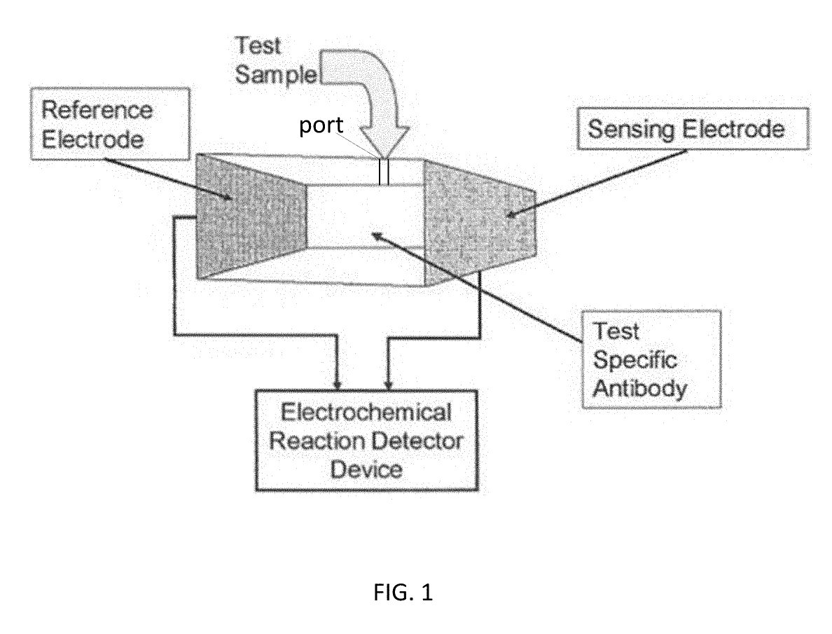

FIG. 1 shows an embodiment of the bio-sensor device;

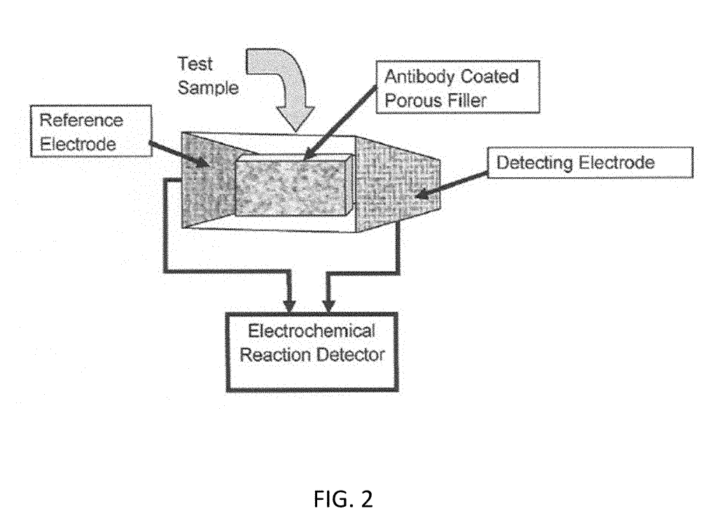

FIG. 2 shows another embodiment of the bio-sensor device;

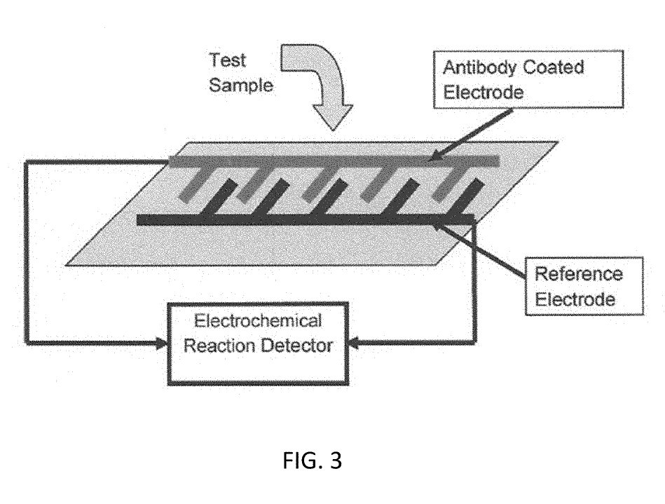

FIG. 3 shows an embodiment of the electrode probes;

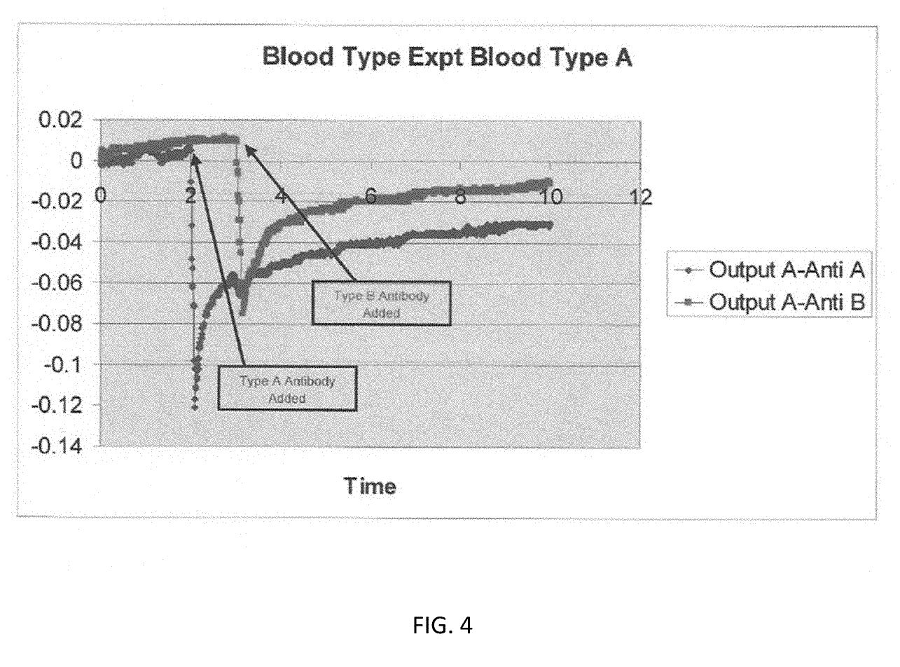

FIG. 4 shows a plot of time versus Output Anti-A and Anti-B for Blood Type Expt Blood Type A;

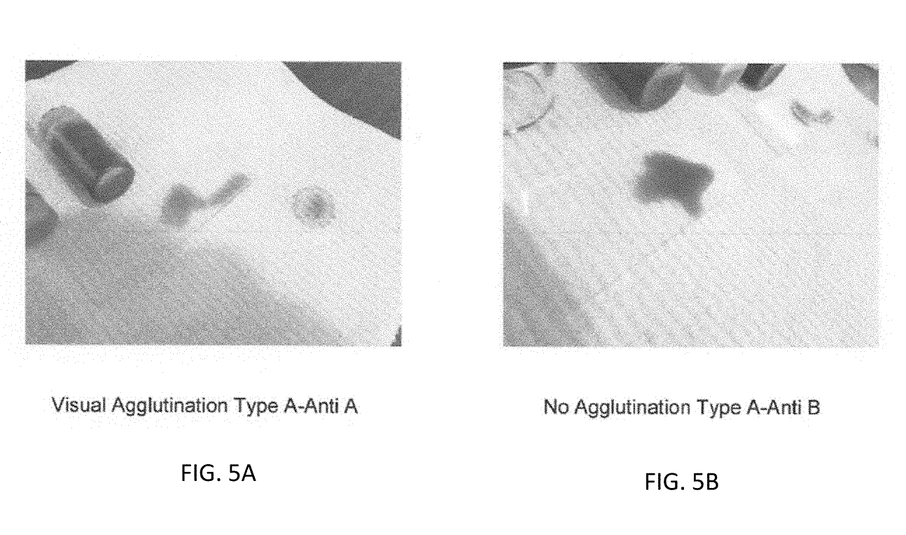

FIGS. 5A and 5B show images of visual Agglutination type Anti-A (FIG. 5A) and Anti-B (FIG. 5B); and

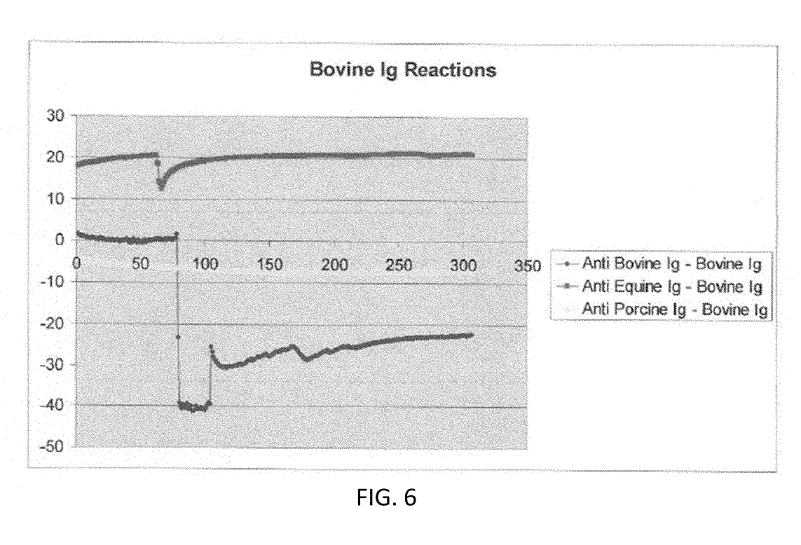

FIG. 6 shows Bovine Ig Reactions; and

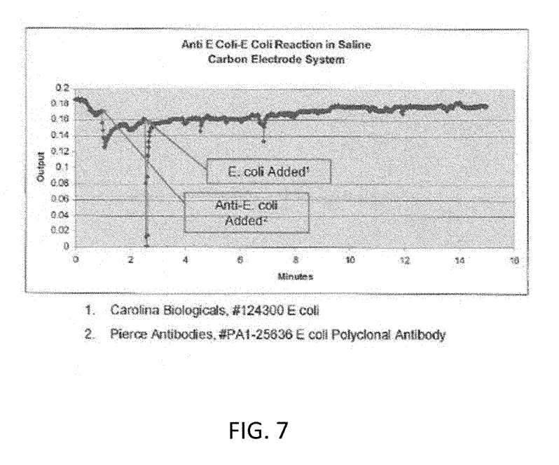

FIG. 7 shows a plot of time versus output of Real Time Detection of Anti E. coli-E. coli Reaction.

### DETAILED DESCRIPTION

A device and methods are described for the real time direct electrochemical detection of Methicillin-Resistant Staphylococcus aureus (MRSA) and Clostridium difficile (C. Diff.) where pathogens are detected as a result of an antigen-antibody reaction. The reaction itself is detected without need for gene or molecular amplification, isolation, separation, or labeling of the products of the reaction. Such devices and methods, involving particular antigen-antibody reactions, are useful in detection of pathogens and contaminants found in infectious disease and in food and water safety applications. The real time speed, specificity, simplicity and broad applicability of the devices and methods described represent improvements to the current art.

We report here, use of a 2 minute direct Antigen-Antibody reaction based test to enable a selective real time screening system (Greene and Yokley, US Application 20100330662 At, Apparatus, System and Method for Consumer Detection of Contaminants in Foodstuffs. Dec. 30, 2010, and subsequent filings, incorporated by reference herein in its entirety).

A selective sensor has been demonstrated based on electrochemical detection of a specific antigen-antibody reaction. This electrochemical method has been previously described as a method of tracking for a wide variety of chemical reactions (W. Tison Wyatt, U.S. Pat. No. 5,749,986, Control of Batching and Curing Processes. May 12, 1998, incorporated by reference herein in its entirety).

The selectivity required in the sensing is determined by detecting a specific fast antibody-antigen reaction. Thus, slow and complex separation, incubation and amplification steps are avoided. This selectivity and specificity are particularly useful in real-time rapid field and consumer level field detection units.

A wide variety of sample configurations can be used on this platform, from probes to flow types. Likewise sample types including, but not limited to saliva, water, washings, homogenates, blood, or other biologic fluids can be tested.

In one embodiment (FIG. 1), the antigen test biosample and the test antibody are added to an appropriately configured electrochemical cell (e.g., via port). This configuration is useful in dealing with infectious outbreak situations. The test chamber and detector device can be contained in the same or different device modules to reduce pathogen handling risk and cost.

In another embodiment (FIG. 2), the antibody is coated onto a porous or fibrous insulating material that is positioned between the electrodes. The sample is then placed in contact with the assembly and the presence or absence of an antigen-antibody reaction is determined by reaction voltage. This configuration is suitable for both small and large sample containers.

In another embodiment, the coated electrode or coated interstitial filler is positioned at the end of a probe or stick, which is connected to a detector directly or by wiring. In this embodiment, the detector is dipped or delivered into the sample. The presence of the specific antigen-antibody reaction is then detected in a similar manner as previously described.

In another favorable embodiment (FIG. 3), the electrodes are laid out in a planar parallel or interdigitated configuration, as shown below. In this embodiment, the reacting antibodies may be present in a solution above the planar array, coated on one electrode, or present in a porous or fibrous carrier located above the array.

Electrodes can be made of of electrically similar conductive materials such as stainless steel, carbon, aluminum, nickel or copper. Non-conductive material can be plated with a layer of electrically conductive matter. The form factor can be plates, wire, wire bundles, foams or other suitable types.

In some embodiments coating one electrode or intracellular filler with a specific antibody, it then is possible to determine the presence of that specific corresponding antigen within the test sample. The selectivity required in the sensing is determined by the antibody-antigen reaction. In this way, slow and complex separation, incubation and amplification steps are avoided. This selectivity is particularly useful in rapid field and consumer level field detection units.

Further, several test chambers can be placed in series so that the sample flows from one test chamber to another, where each test chamber contains an antibody, either coated or free, specific to a different biological material or pathogen of interest. In this way, for example, a single vegetable homogenate sample could be tested for the presence of both Salmonella and E. coli in a single test pass.

The configurations described permit real time detection of specific antigens with high sensitivity. The ultimate sensitivity of the method is determined by the antigen and antibody concentrations as well as the specificity of the antibody against the pathogen or contaminant. The reaction is rapid, without the need for long sample incubation or the use of additional reagents.

It is also possible to provide mixtures of antibodies in the chamber or probe. This arrangement would allow for the detection of multiple strains of the same pathogen, or for mixtures of antigens that might be characteristic of a condition of interest. (See for example the recent report linking pancreatic cancer risk with a characteristic group of oral bacteria. http:1/news.brown.edu/pressreleases/2012/09/periodontic . Dominique S Michaud, Jacques Izard, Charlotte S Wilhelm-Benartzi, Doo-Ho You, Verena A Grote. Anne Tjczmneland. Christina C Dahm, Kim Overvad. Mazda Jenab, Veronika Fedirko, Marie Christine Boutron-Ruault, Frangoise Clavei-Chapelon, Antoine Racine, Rudolf Kaaks, Heiner Boeing, Jana Foerster. Antonia Trichopoulou, Pagona Lagiou. Dimitrios Trichopoulos, Carlotta Sacerdote. Sabina Sieri, Domenico Palli, Rosario Tumino, Salvatore Panico, Peter D Siersema. Petra H M Peeters, Eiliv Lund, Aurelio Barricarte, Jose-Maria Huerta, Esther Molina-Montes, Miren Dorronsoro, J Ramon Quiros, Eric J Duell, Weimin Ye, Malin Sund, Bjorn Lindkvist, Dorthe Johansen, Kay-Tee Khaw, Nick Wareham, Ruth C Travis, Paolo Vineis, H Bas Bueno-de-Mesquita, Elio Riboli. Plasma antibodies to oral bacteria and risk of pancreatic cancer in a large European prospective cohort study. Gut, 18 Sep. 2012 D01: 10.1136/gutjnl-2012-303006)

Reaction chambers made of various materials and in a variety of sizes most preferably glass, silicon or a polymeric material. For quick field tests for contamination of food or drinking water samples, a small test chamber of 1-10 ml might be most appropriate. When testing samples of meat or vegetables for contamination, larger sample containers, designed to hold between 10 to 100 ml of a liquefied preparation may be better suited.

In a preferred embodiment, the reaction chamber is molded in two halves which can be snapped together to form a reaction chamber. In this embodiment, the antibody coated electrode is produced and packaged separately in one half chamber. The reference electrode is assembled and attached to the other half. Manufacturing is therefore simplified. It is then possible to mix and match sensors for various antigens from the smallest number of parts.

This described invention can be at a modular breadboard stage of development. Several integrated product configurations of the probes/chambers and the intermediate electronics and the computer/tabletlsmartphone data logging device, as required by the specific application are possible. The form factor can be plates, wire, wire bundles, foams or other suitable types. These might include hand held, and devices where the test chamber unit contains a wireless communication module so that the chamber is never touched by anyone other than the subject providing the sample.

Electrodes can be made of any of several electrically similar conductive materials such as stainless steel, carbon, aluminum, nickel or copper, gold or silver, tungsten, and any of their conductive compounds or alloys.

Several examples are described below:

### EXAMPLE ONE

Real Time Detection of Blood Typing Antigen-Antibody Reaction with Visual Confirmation via Agglutination

ABO Blood type is determined by antigens on the surface of red blood cells. When exposed to a specific antibody, the blood cells will agglutinate. For example, a drop of Type A blood mixed with Anti-A antibody will develop a granular appearance on a glass slide as the cells agglutinate. A drop of Type B blood mixed with the same antibody will remain homogeneous in appearance.

Using Carolina Biological Supply Blood Typing Kit #700122, we conducted an experiment in which we mixed Type A blood with Type A Antibody, Type A blood with Type B antibody, Type B blood with Type B Antibody, and Type B blood with Type A Antibody. As expected, we observed agglutination only with S-Anti B and A-Anti A.

Once the activity of the samples was confirmed, we did the same experiment using the technology presented in this proposal. The results are illustrated in the charts and photographs shown below (FIGS. 4-5B), indicating expected reactions visually and by change in electrical potential.

### EXAMPLE TWO

Selective Real Time Sensor Detection: Antigen-Antibody Solution Reaction

Three Goat Anti-albumin samples for equine, bovine, and porcine albumin were prepared in distilled water in individual vials. Each sample vial was equipped with an ElectroImmune sensor probe. Each sample, in turn was connected to the ElectroImmune sensor. Bovine Albumin supplied in the same kit was added to the chamber.

The results of the experiment are summarized in Table 1, and the sensor output traces are shown below (FIG. 6).

TABLE 1
Antigen-Antibody Detection via Sensor Reaction

| Antibody | Antigen | Result |
|---|---|---|
| Goat Anti-Bovine Albumin | Bovine 1 g Albumin | Immediate |
| | | Reaction |
| | | on Mixing |
| Goat Anti-Equine Albumin | Bovine 1 g Albumin | No Reaction |
| Goat Anti-Porcine Albumin | Bovine 1 g Albumin | No Reaction |

Thus, specificity of the method is demonstrated.

### EXAMPLE THREE

A bacterial detection demonstration was conducted as follows. A suspension of polyclonal Escheria coli antibody [Pierce Antibodies, #PA125636] in saline was challenged with a commercial E. coli [Carolina Biologicals, #124300] solution in an electrochemical test chamber. The electrochemical reaction trace was recorded on a PC level platform.

The antigen-antibody reaction produced a real time electrochemical displacement signal which was readily detected and repeatable (FIG. 7).

Embodiment can be a breadboard level device which will detect antigen-antibody reactions in real time by electrochemical detection, suitable for detection of a bacterial infection pathogen ex vivo. This configuration is based on our previous antigen-antibody demonstration work. Further, as described above, numerous configurations to expose the sample material to the appropriate antibody of interest are possible and will depend on the specific needs for the test involved. Hospital and wound derived infections such as Methicillin-resistant Staphylococcus aureus [MRSA] and Clostridium difficile (C. difficile), a bacterium that causes diarrhea and more serious intestinal conditions such as colitis, are a particular concern.

Strains that are oxacillin and methicillin resistant, historically termed methicillin-resistant S. aureus (MRSA), are resistant to all B-lactam agents, including cephalosporins and carbapenems, although they may be susceptible to the newest class of MRSA-active cephalosporins (e.g, ceftaroline). Strains of MRSA causing healthcare-associated infections often are multiply resistant to other commonly used antimicrobial agents, including erythromycin, clindamycin, fluoroquinolones and tetracycline, while strains causing community-associated infections are often resistant only to B-lactam agents and erythromycin, may be resistant to fluoroquinolones. Since 1996, MRSA strains with decreased susceptibility to vancomycin (minimum inhibitory concentration [MIC], 4-8 j..1 g/ml) and strains fully resistant to vancomycin (MIC 32 j..1 g/ml) have been reported (https://www.cdc.gov/mrsa/lab/index.html)

Table 2 presents a summary of types of clinical of MRSA tests, shown here for reference (E. Sturenburg, GMS German Medical Science 2009, Vol. 7,ISSN 1612-3174.).

TABLE 2

| | | | | | | | |
|---|---|---|---|---|---|---|---|
| | | | | | Systems can | | |
| | | | Turn-around | Co [symbol] | be used with | Author | |
| Test | Distributor | Test Concept | time | swab | swabs from | year [Ref] | Performance data |

I Single-locus PCR: SCCmec PCR: suitable for point-of-care testing

| | | | | | | | |
|---|---|---|---|---|---|---|---|
| GeneXpert | Genezyme | GeneXpert DX Cycler; | 75 min | 25-35 ϵ | nose | Ceph [symbol] | Sens: 86.3% Spec 9 [symbol] .9% |
| MRSA | V [symbol] ch | single-use cartridges | | | | 2007 [ [symbol] ]: | PPV: 80.6% NPV: 98.6% |
| | | containing freeze dried | | | | Roseney | Sens: 90% Spec: 97% |
| | | beads with all reagents | | | | 2008 [ [symbol] ] | PPV: 86% NPV: 98% |
| | | required for PCR | | | | | |

II Single-locus PCR: SCCmec-PCR

| | | | | | | | |
|---|---|---|---|---|---|---|---|
| RD GeneOnm | Electon | timerCycler | <2 h | 20 ϵ | nose | Hule [symbol] | Sens: 96.7% Spec: [symbol] .4% |
| MRSA | Dickinson | | | | | 2004 [28]: | PPV: na NPV: na |
| | | | | | | Desjardins | Sens: 96% Spec: 96% |
| | | | | | | 2006 [34]: | PPV: 90% NPV: [symbol] % |
| | | | | | | de San | Sens: [symbol] % Spec [symbol] % |
| | | | | | | 2007 [33]: | PPV: [symbol] % NPV: [symbol] % |
| | | | | | | Boyce | Sens: 100% Spec: [symbol] % |
| | | | | | | 2008 [32]: | PPV: [symbol] .8% NPV: 100% |
| | | | | | | Oberdorfer | Sens: 100% Spec: 98.8% |
| | | | | | | 2008 [31] | PPV: [symbol] % NPV: 100% |
| GenoType | Rain | Conventional cycling | 4-5 h | [symbol] ϵ | nose, throat, | Holfelder | Sens: 9 [symbol] -9 [symbol] % Spec: 99% |
| MRSA Direct | Lifesciences | followed by [symbol] -bot | | | hairline, | 2006 [30] | PPV: [symbol] -88% NPV: 99% |
| | | assay | | | wounds | | |

III Multilocus PCR: merA plus S. aureus marker gene plus CoNS marker genes

| | | | | | | | |
|---|---|---|---|---|---|---|---|
| hyplex | BAG | (mecA → S. aureus/ | 4-5 h | 10 ϵ | swabs (not | Leven | Sens: 83% Spec: [symbol] % |
| Staphylo | | [symbol] . epidermidis/ S. | | | specified | 2007 [27]; | PPV: 83% NPV: 98% |
| Realek | | [symbol] -specific [symbol] / | | | respiratory | Koelemann | Sens: 100% Spec: 95% |
| | | conventional cycling | | | [symbol] | 2005 [26] | PPV: 61% NPV: 100% |
| | | followed by enzyme- | | | | | |
| | | immuno assay | | | | | |
| LightCycler | Roche | (mecA * 16S-23S ITS | <2 h | 15-20 ϵ | swabs (not | Kols | Sens: [symbol] % Spec: 97% |
| Staphylocoocus/ | Diagnostics | sequence with | | | specified) | 2005 [25] | PPV: 60% NPV: 99.4% |
| MRSA Kit | | melting point analysis | | | | | |
| | | of the species)/ | | | | | |
| | | LightCycler | | | | | |

IV Rapid culture/without any nucleic acid amplification

| | | | | | | | |
|---|---|---|---|---|---|---|---|
| 3M BacLife | 3M | Selective broth | 5 h | 10 ϵ | nose, groin | O'Hara | Sens: 94.8% Spec: 96.9% |
| Rapid MRSA | Company | enrichment > magnetic | | ( [symbol] | | 2007 [19]; | PPV: na NPV: na |
| Test | | micro [symbol] separation > | | sample/ | | Cohen | Sens: [symbol] % Spec: [symbol] % |
| | | [symbol] lysis > bio [symbol] | | day | | 2007 [19] | PPV: na NPV: na |
| | | measurement | | | | | |

Abbreviations/annotations
GeneXpert DX systems, fully automated platform for real-time PCR cycling, only little operator handling/knowledge required, works with single-use disposable cartridges containing at PCR reagents required; 16S-23S ITS, 16S-23S IDNA [symbol] nal transcribed spacer region, CoNS Coagulase-negative staphylo [symbol] LightCycles, special instrument for real-time PCR cycling, mecA gene conferring [symbol] resistance in staphylo [symbol] ; SmartCycler, special instrument for real time PCR cycling, only little operator handling/knowledge required; SCCmec [symbol] , DNA sequences in the region of the open reading frame [symbol] the staphyl [symbol] chromosome [symbol] ) integrates into the S. aureus chromosome. SCCmec carries the resistance determinant mecA.
NPV, negative predictive value; Sens, sensistivity; Spec. specifically; PPV, posistive predictive value; na not avaible [symbol]
indicates data missing or illegible when filed

Clostridium difficile (C. diffici/e) is a bacterium that is related to the bacteria that cause tetanus and botulism. The C. difficile bacterium has two forms, an active, infectious form that cannot survive in the environment for prolonged periods, and an inactive, “noninfectious” form, called a spore, that can survive in the environment for prolonged periods. Although spores cannot cause infection directly, when they are ingested they transform into the active, infectious form.

C. difficile spores are found frequently in: hospitals, nursing homes, extended care facilities, and nurseries for newborn infants.

They can be found on: bedpans, furniture, toilet seats, linens, telephones, stethoscopes, fingernails, rings, jewelry), floors, infants' rooms, and diaper pails.

They even can be carried by pets. Thus, these environments are a ready source for infection with C. difficile (https://www.medicinenet.com/clostridium difficile colitis/article.htm).

Antibiotic-associated (C. difficile) colitis is an infection of the colon caused by C. difficile that occurs primarily among individuals who have been using antibiotics. C. difficile infections are commonly acquired during hospital stays, infecting approximately 1% of patients admitted to hospitals in the United States. C. difficile may also be acquired in the community, however.

It is the most common infection acquired by patients while they are in the hospital. More than half a million C. difficile infections occur in hospitals in the US each year, with about 300,000 occurring while in the hospital or shortly after hospitalization. After a stay of only two days in a hospital, 10% of patients will develop infection with C. difficile. C. difficile also may be acquired outside of hospitals in the community. It is estimated that about 200,000 infections with C. difficile occur in the community unrelated to hospitalization each year in the U.S.

Diagnosis of Clostridium difficile infection is based on clinical presentation and laboratory tests. Although numerous laboratory methods are now available, the diagnosis of C. difficile infection remains challenging. Nucleic acid amplification tests (NAATs) are the most recent marketed methods. These methods detect genes for toxins A and/or B. They are very sensitive compared with the reference method (toxigenic culture). However, these test require specialized equipment and are not rapid enough for use in the field or in a physician's office.

Those skilled in the art to which this application relates will appreciate that other and further additions, deletions, substitutions and modifications may be made to the described embodiments.

What is claimed is:

1. A bio-sensor device for the electro-chemical detection of a bacterial pathogen, the device comprising:
a sample chamber including electrical probes to detect pathogenic antigens in a sample containing the bacterial pathogen, wherein the electrical probes detect a reaction voltage corresponding to an antigen-antibody reaction occurring when the pathogenic antigens come into contact with an antibody specific for pathogenic antigens present in a reaction medium in the sample chamber and contacted by the electrical probes; and
an electronic data module to detect and process electrical signals from the conductive electrical probes corresponding to an amount of the antigen present in the sample, wherein the reaction voltage is detected at the time of the reaction.

2. The detection device of claim 1, wherein the bacterial pathogen is methicillin-Resistant Staphylococcus aureus.

3. The detection device of claim 1, wherein the bacterial pathogen is Clostridium difficle.

4. The detection device of claim 1, wherein the bacterial pathogen includes methicillin-Resistant Staphylococcus aureus and Clostridium difficle.

5. The device of claim 1, wherein the pathogen is detected ex vivo from a patient derived sample, including but not limited to blood, saliva, wound exudates, and stool.

6. The device of claim 1, wherein the pathogen is detected from direct testing of a, swab, or washings from a surface.

7. The device of claim 7, wherein the surface is an epidermis of an organism.

8. The device of claim 7, wherein the surface is a potentially contaminated non-biologic surface including a counter-top or synthetic athletic playing surface.

9. The device of claim 7, wherein the surface is a wound dressing.

10. The device of claim 1, wherein the device is configured as a real-time detection device for detecting the presence of the pathogenic antigens, and the real-time detection device is self-contained and field-applicable, not requiring external equipment or highly trained laboratory personnel.

11. The device of claim 10, wherein the electrical probes of the real-time detection device are configured to respond to electrochemical antigen-antibody events corresponding to the antigen-antibody reaction within 60 seconds of the sample containing the pathogenic antigens and the antibody-containing reaction medium in the sample chamber becoming in contact with each other.

12. The device of claim 10, wherein the real-time detection device is configured for direct electrochemical reaction detection of the antigen-antibody reaction.

13. The device of claim 10, wherein the real-time detection device is not sensitive to detection of reaction products of the antigen-antibody reaction.

14. The device of claim 1, wherein the antibody specific for the pathogenic antigens is coated onto one or more of the sensing electrodes.

15. The device of claim 1, wherein the antibody specific for the pathogenic antigens is coated or adsorbed onto a passive filler located within the sample chamber.

16. The device of claim 1, wherein the sample chamber includes a port for introducing a liquid reagent containing the antibody specific for the pathogenic antigens.
